(19) United States
(12) Patent Application Publication
(10) Pub. No.: US 2016/0227778 A1
(43) Pub. Date: Aug. 11, 2016

(54) SEED DRESSING FOR CONTROLLING PHYTOPATHOGENIC FUNGI

(71) Applicant: BAYER INTELLECTUAL PROPERTY GMBH, MONHEIM AM RHEIN (DE)

(72) Inventors: GEOFF KNEEN, CHAPEL HILL, NC (US); ANNE SUTY-HEINZE, LANGENFELD (DE); PETER DAHMEN, NEUSS (DE); YASUO ARAKI, TOCHIGI (JP); TAKUMA SHIGYO, KANAGAWA (JP); HANS-LUDWIG ELBE, WUPPERTAL (DE)

(21) Appl. No.: 15/131,759

(22) Filed: Apr. 18, 2016

Related U.S. Application Data
(63) Continuation of application No. 11/629,982, filed on Nov. 1, 2007, now abandoned.

(30) Foreign Application Priority Data
Jun. 21, 2004 (DE) 10 2004 029 972.2

(51) Int. Cl. A01N 43/56; C07D 231/16; C07D 409/12

(52) U.S. Cl. CPC A01N 43/56; C07D 409/12; C07D 231/16

(57) ABSTRACT

The present invention relates to the use of fungicidally active carboxamides for seed treatment, for corresponding seed dressings comprising these carboxamides, to a process for controlling phytopathogenic fungi by treating the seed with fungicidally active carboxamides, and also to seed which has been treated with the fungicidally active carboxamides.

The present invention relates to the use of fungicidally active carboxamides for seed treatment, for corresponding seed dressings comprising these carboxamides, to a process for controlling phytopathogenic fungi by treating the seed with fungicidally active carboxamides, and also to seed which has been treated with the fungicidally active carboxamides.

It is already known that certain carboxamides have fungicidal properties. Thus, for example, N-[2-(1,3-dimethylbutyl)thiophen-3-yl]-1-methyl-3-(trifluoromethyl)-1H-pyrazole-4-carboxamide (EP-A 0 737 682), N-[2-(1,3-dimethylbutyl)phenyl]-5-fluoro-1,3-dimethyl-1H-pyrazole-4-carboxamide (WO 03/010149) and N-[2-(1,3-dimethylbutyl)phenyl]-1-methyl-4-trifluoromethyl-1H-pyrrolecarboxamide (WO 02/38542) are each known to be suitable for controlling phytopathogenic fungi by foliar application as sprays. The use of these compounds for treating seed for protection against attack by phytopathogenic fungi has hitherto not been disclosed. In particular, the use of these compounds as seed dressing for protection against attack by Pyrenophora, Rhizoctonia, Tilletia and Ustilago species is new.

It has now been found that carboxamides of the general formula (I)

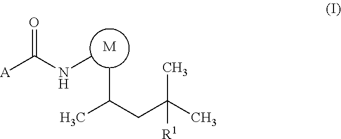

in which
M represents one of rings M1 to M4 below

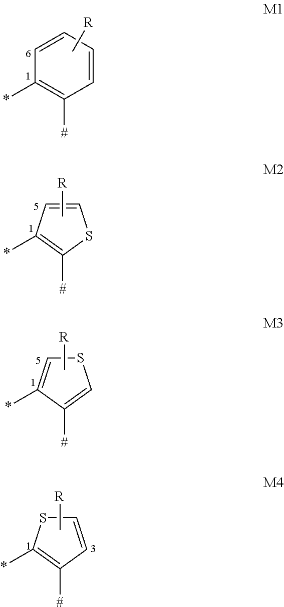

where the bond marked “*” is attached to the amide and the bond marked “#” is attached to the alkyl chain,
R represents hydrogen, fluorine, chlorine or methyl,
R¹ represents hydrogen, halogen, C₁-C₃-alkyl or C₁-C₃-haloalkyl having 1 to 7 fluorine, chlorine and/or bromine atoms,
A represents one of the radicals A1 to A8 below:

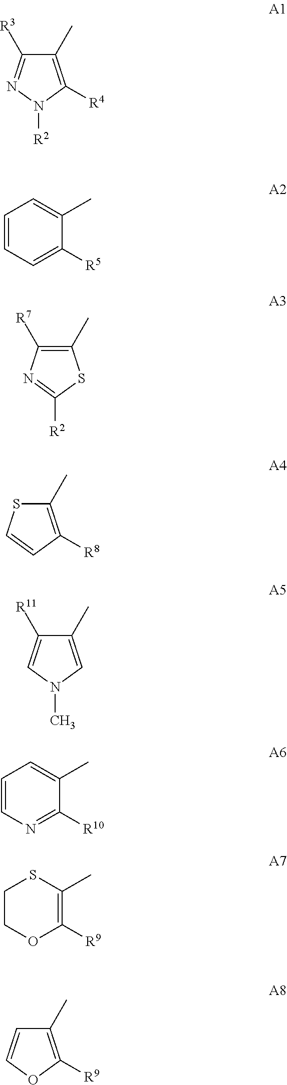

R² represents C₁-C₃-alkyl,
R³ represents hydrogen, halogen, C₁-C₃-alkyl or C₁-C₃-haloalkyl having 1 to 7 fluorine, chlorine and/or bromine atoms,
R⁴ represents hydrogen, halogen or C₁-C₃-alkyl,
R⁵ represents halogen, C₁-C₃-alkyl or C₁-C₃-haloalkyl having 1 to 7 fluorine, chlorine and/or bromine atoms,
R⁶ represents hydrogen, halogen, C₁-C₃-alkyl, amino, mono- or di(C₁-C₃-alkyl)-amino,
R⁷ represents hydrogen, halogen, C₁-C₃-alkyl or C₁-C₃-haloalkyl having 1 to 7 fluorine, chlorine and/or bromine atoms,
R⁸ represents halogen, C₁-C₃-alkyl or C₁-C₃-haloalkyl having 1 to 7 fluorine, chlorine and/or bromine atoms,
R⁹ represents halogen, C₁-C₃-alkyl or C₁-C₃-haloalkyl having 1 to 7 fluorine, chlorine and/or bromine atoms,
R¹⁰ represents hydrogen, halogen, C₁-C₃-alkyl or C₁-C₃-haloalkyl having 1 to 7 fluorine, chlorine and/or bromine atoms,
R¹¹ represents hydrogen, halogen, C₁-C₃-alkyl or C₁-C₃-haloalkyl having 1 to 7 fluorine, chlorine and/or bromine atoms,
   are highly suitable for treating (dressing) seed against attack by phytopathogenic fungi.

Surprisingly, by using the active compounds according to the invention as seed dressing, some phytopathogenic fungi can be controlled considerably more effectively than by spray treatment in the case of foliar application.

The formula (I) provides a general definition of the carboxamides which can be used for treating seed. Preference is given to using carboxamides of the formula (I) in which the radicals are as defined below.
M preferably represents M-1.
M furthermore preferably represents M-2.
R preferably represents hydrogen.
R, if M represents M-1, furthermore preferably represents fluorine, where fluorine is located particularly preferably in the 4-, 5- or 6-position, very particularly preferably in the 4- or 6-position, especially in the 4-position, of the anilide radical.
R, if M represents M-1, furthermore preferably represents chlorine, where chlorine is particularly preferably located in the 4-, 5- or 6-position, very particularly preferably in the 4- or 6-position, especially in the 4-position, of the anilide radical.
R, if M represents M-1, furthermore preferably represents methyl, where methyl is particularly preferably located in the 3-position of the anilide radical [cf. above formula (I)].
R, if M represents M-2, M-3 or M-4 furthermore preferably represents methyl, where methyl is particularly preferably located in the 5-position (M-2, M-3) or in the 3-position (M-4).
R¹ preferably represents hydrogen, fluorine, chlorine, methyl, ethyl, n-propyl, isopropyl, monofluoromethyl, difluoromethyl, trifluoromethyl, monochloromethyl, dichloromethyl or trichloromethyl.
R¹ particularly preferably represents hydrogen, fluorine, chlorine, methyl, ethyl or trifluoromethyl.
R¹ very particularly preferably represents hydrogen or methyl.
R¹ especially represents hydrogen.
R¹ furthermore especially represents methyl.
A preferably represents the radicals A1, A2, A3, A4 or A5.
A particularly preferably represents the radicals A1, A2, A3 or A5.
A very particularly preferably represents the radical A1.
A furthermore very particularly preferably represents the radical A2.
A furthermore very particularly preferably represents the radical A3.
A furthermore very particularly preferably represents the radical A5.
R² preferably represents methyl, ethyl, n-propyl or isopropyl.
R² particularly preferably represents methyl or isopropyl.
R² very particularly preferably represents methyl.
R³ preferably represents iodine, methyl, difluoromethyl or trifluoromethyl.
R³ particularly preferably, represents methyl, difluoromethyl or trifluoromethyl.
R³ very particularly preferably represents methyl.
R⁴ preferably represents hydrogen, fluorine, chlorine or methyl.
R⁴ particularly preferably represents hydrogen or fluorine.
R⁴ very particularly preferably represents fluorine.
R⁵ preferably represents chlorine, bromine, iodine, methyl, difluoromethyl or trifluoromethyl.
R⁵ particularly preferably represents iodine, difluoromethyl or trifluoromethyl.
R⁵ very particularly preferably represents iodine or trifluoromethyl.
R⁶ preferably represents hydrogen, chlorine, methyl or amino.
R⁶ particularly preferably represents methyl or amino.
R⁶ very particularly preferably represents methyl.
R⁷ preferably represents hydrogen, chlorine, methyl, difluoromethyl or trifluoromethyl.
R⁷ particularly preferably represents chlorine, methyl, difluoromethyl or trifluoromethyl.
R⁷ very particularly preferably represents methyl, difluoromethyl or trifluoromethyl.
R⁸ preferably represents chlorine, bromine, iodine, methyl or trifluoromethyl.
R⁸ particularly preferably represents bromine, iodine, methyl or trifluoromethyl.
R⁸ very particularly preferably represents bromine, iodine or methyl.
R⁹ preferably represents chlorine, bromine, iodine, methyl, isopropyl or trifluoromethyl.
R⁹ particularly preferably represents methyl, isopropyl or trifluoromethyl.
R⁹ very particularly preferably represents methyl or trifluoromethyl.
R¹⁰ preferably represents fluorine, chlorine, methyl or trifluoromethyl.
R¹⁰ particularly preferably represents chlorine or trifluoromethyl.
R¹⁰ very particularly preferably represents chlorine.
R¹¹ preferably represents chlorine, iodine, methyl, difluoromethyl or trifluoromethyl.
R¹¹ particularly preferably represents methyl, difluoromethyl or trifluoromethyl.
R¹¹ very particularly preferably represents trifluoromethyl.

Very particularly preferably suitable for seed treatment are carboxamides of the formula (I-a)

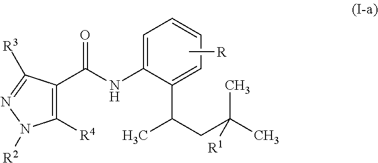

in which R, R¹, R², R³ and R⁴ are as defined above and R¹ represents in particular hydrogen or methyl.

Very particularly preferably suitable for seed treatment are furthermore carboxamides of the formula (I-b)

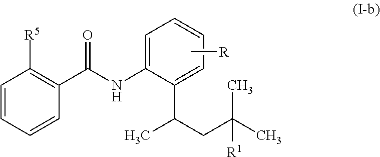

in which R, R¹ and R⁵ are as defined above and R¹ represents in particular hydrogen or methyl.

Very particularly preferably suitable for seed treatment are furthermore carboxamides of the formula (I-c)

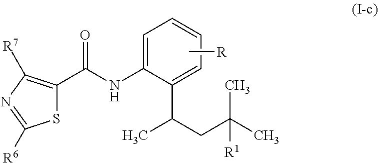

in which R, R¹, R⁶ and R⁷ are as defined above and R¹ represents in particular hydrogen or methyl.

Very particularly preferably suitable for seed treatment are furthermore carboxamides of the formula (I-d)

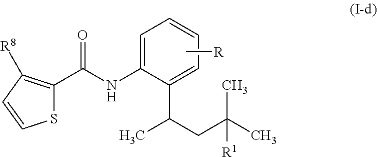

in which R, R¹ and R⁸ are as defined above and R¹ represents in particular hydrogen or methyl.

Very particularly preferably suitable for seed treatment are furthermore carboxamides of the formula (I-e)

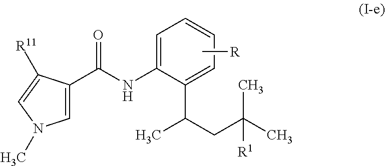

in which R, R¹ and R¹¹ are as defined above and R¹ represents in particular hydrogen or methyl.

Very particularly preferably suitable for seed treatment are furthermore carboxamides of the formula (I-f)

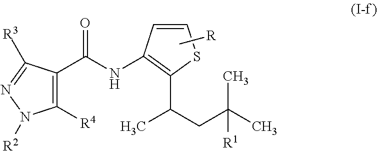

in which R, R¹, R², R³ and R⁴ are as defined above and R¹ represents in particular hydrogen or methyl.

Very particularly preferably suitable for seed treatment are furthermore carboxamides of the formula (I-g)

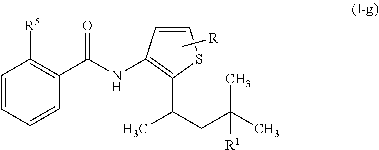

in which R, R¹ and R⁵ are as defined above and R¹ represents in particular hydrogen or methyl.

Very particularly preferably suitable for seed treatment are furthermore carboxamides of the formula (I-h)

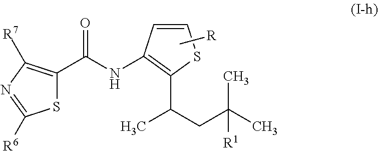

in which R, R¹, R⁶ and R⁷ are as defined above and R¹ represents in particular hydrogen or methyl.

Very particularly preferably suitable for seed treatment are furthermore carboxamides of the formula (I-j)

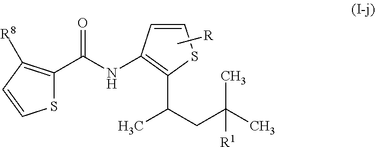

in which R, R¹ and R⁸ are as defined above and R¹ represents in particular hydrogen or methyl.

Very particularly preferably suitable for seed treatment are furthermore carboxamides of the formula (I-k)

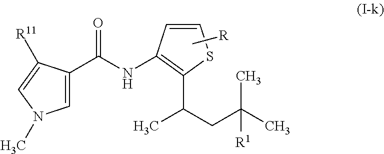

in which R, R¹ and R¹¹ are as defined above and R¹ represents in particular hydrogen or methyl.

The formula (I) encompasses in particular the following carboxamides which can preferably be used for seed treatment:
(I-1) N-[2-(1,3-dimethylbutyl)phenyl]-1,3-dimethyl-1H-pyrazole-4-carboxamide of the formula

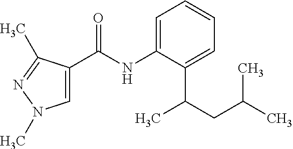 [SMILES: Cc1nn(C)cc1C(=O)Nc1ccccc1C(C)CC(C)C]

(known from JP-A 10-251240)
(I-2) N-[2-(1,3-dimethylbutyl)phenyl]-5-fluoro-1,3-dimethyl-1H-pyrazole-4-carboxamide of the formula

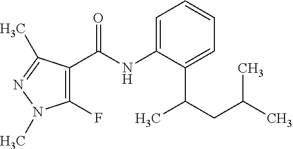 [SMILES: Cc1nn(C)c(F)c1C(=O)Nc1ccccc1C(C)CC(C)C]

(known from WO 03/010149)
(I-3) N-[2-(1,3-dimethylbutyl)phenyl]-5-chloro-1,3-dimethyl-1H-pyrazole-4-carboxamide of the formula

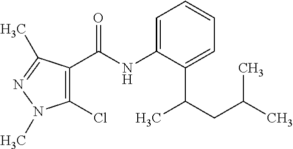 [SMILES: Cc1nn(C)c(Cl)c1C(=O)Nc1ccccc1C(C)CC(C)C]

(known from JP-A 10-251240)
(I-4) 3-(difluoromethyl)-N-[2-(1,3-dimethylbutyl)phenyl]-1-methyl-1H-pyrazole-4-carboxamide of the formula

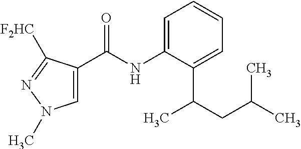 [SMILES: CC(C)CC(C)c1ccccc1NC(=O)c1cn(C)nc1C(F)F]

(known from JP-A 10-310577)
(I-5) 3-(trifluoromethyl)-N-[2-(1,3-dimethylbutyl)phenyl]-5-fluoro-1-methyl-1H-pyrazole-4-carboxamide of the formula

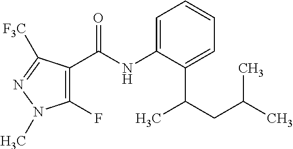 [SMILES: CC(C)CC(C)c1ccccc1NC(=O)c1c(C(F)(F)F)nn(C)c1F]

(known from DE-A 103 03 589)
(I-6) 3-(trifluoromethyl)-N-[2-(1,3-dimethylbutyl)phenyl]-5-chloro-1-methyl-1H-pyrazole-4-carboxamide of the formula

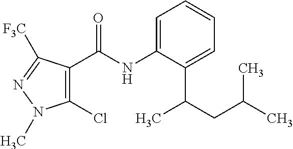 [SMILES: CC(C)CC(C)c1ccccc1NC(=O)c1c(C(F)(F)F)nn(C)c1Cl]

(known from JP-A 10-251240)
(I-7) 1,3-dimethyl-N-[2-(1,3,3-trimethylbutyl)phenyl]-1H-pyrazole-4-carboxamide of the formula

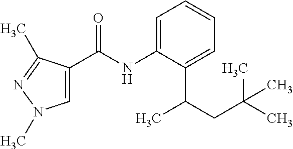 [SMILES: Cc1nn(C)cc1C(=O)Nc1ccccc1C(C)CC(C)(C)C]

(known from JP-A 10-251240)
(I-8) 5-fluoro-1,3-dimethyl-N-[2-(1,3,3-trimethylbutyl)phenyl]-1H-pyrazole-4-carboxamide of the formula

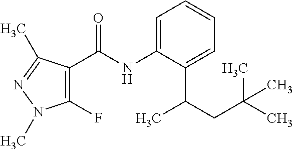 [SMILES: Cc1nn(C)c(F)c1C(=O)Nc1ccccc1C(C)CC(C)(C)C]

(known from WO 03/010149)
(I-9) 3-(difluoromethyl)-1-methyl-N-[2-(1,3,3-trimethylbutyl)phenyl]-1H-pyrazole-4-carboxamide of the formula

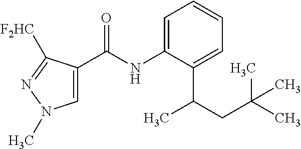 [SMILES: CC(CC(C)(C)C)c1ccccc1NC(=O)c1cn(C)nc1C(F)F]

(known from DE-A 103 52 082)
(I-10) 3-(trifluoromethyl)-1-methyl-N-[2-(1,3,3-trimethylbutyl)phenyl]-1H-pyrazole-4-carboxamide of the formula

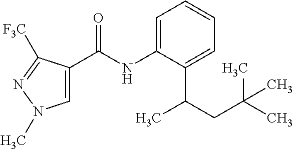 [SMILES: CC(CC(C)(C)C)c1ccccc1NC(=O)c1cn(C)nc1C(F)(F)F]

(known from DE-A 103 52 082)
(I-11) 3-(trifluoromethyl)-5-fluoro-1-methyl-N-[2-(1,3,3-trimethylbutyl)phenyl]-1H-pyrazole-4-carboxamide of the formula

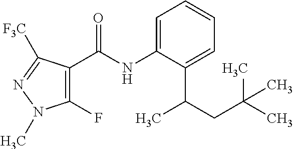 [SMILES: CC(CC(C)(C)C)c1ccccc1NC(=O)c1c(C(F)(F)F)nn(C)c1F]

(known from DE-A 103 03 589)
(I-12) 3-(trifluoromethyl)-5-chloro-1-methyl-N-[2-(1,3,3-trimethylbutyl)phenyl]-1H-pyrazole-4-carboxamide of the formula

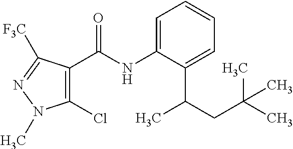 [SMILES: CC(CC(C)(C)C)c1ccccc1NC(=O)c1c(C(F)(F)F)nn(C)c1Cl]

(known from JP-A 10-251240)
(I-13) N-[2-(1,3-dimethylbutyl)phenyl]-2-iodobenzamide of the formula

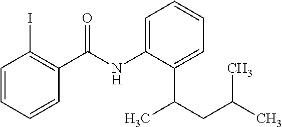 [SMILES: CC(C)CC(C)c1ccccc1NC(=O)c1ccccc1I]

(known from DE-A 102 29 595)
(I-14) 2-iodo-N-[2-(1,3,3-trimethylbutyl)phenyl]benzamide of the formula

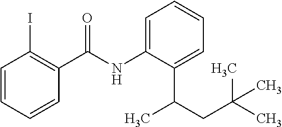 [SMILES: CC(CC(C)(C)C)c1ccccc1NC(=O)c1ccccc1I]

(known from DE-A 102 29 595)
(I-15) N-[2-(1,3-dimethylbutyl)phenyl]-2-(trifluoromethyl)benzamide of the formula

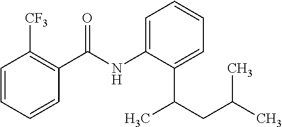 [SMILES: CC(C)CC(C)c1ccccc1NC(=O)c1ccccc1C(F)(F)F]

(known from DE-A 102 29 595)
(I-16) 2-(trifluoromethyl)-N-[2-(1,3,3-trimethylbutyl)phenyl]benzamide of the formula

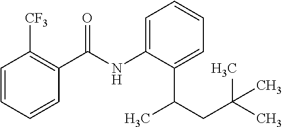 [SMILES: CC(CC(C)(C)C)c1ccccc1NC(=O)c1ccccc1C(F)(F)F]

(known from DE-A 102 29 595)
(I-17) 3-(difluoromethyl)-N-[2-(1,3-dimethylbutyl)phenyl]-1-methyl-1H-pyrrole-4-carboxamide of the formula

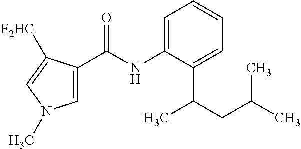 [SMILES: CC(C)CC(C)c1ccccc1NC(=O)c1cn(C)cc1C(F)F]

(known from WO 02/38542)
(I-18) 3-(trifluoromethyl)-N-[2-(1,3,3-trimethylbutyl)phenyl]-1-methyl-1H-pyrrole-4-carboxamide of the formula

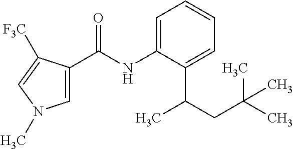 [SMILES: CC(CC(C)(C)C)c1ccccc1NC(=O)c1cn(C)cc1C(F)(F)F]

(I-19) 3-(difluoromethyl)-N-[2-(1,3,3-dimethylbutyl)phenyl]-1-methyl-1H-pyrrole-4-carboxamide of the formula

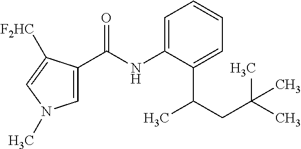 [SMILES: CC(CC(C)(C)C)c1ccccc1NC(=O)c1cn(C)cc1C(F)F]

(I-20) 3-(trifluoromethyl)-N-[2-(1,3-dimethylbutyl)phenyl]-1-methyl-1H-pyrrole-4-carboxamide of the formula

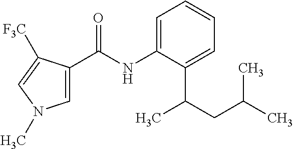 [SMILES: CC(C)CC(C)c1ccccc1NC(=O)c1cn(C)cc1C(F)(F)F]

(known from WO 02/38542)
(I-21) N-[2-(1,3-dimethylbutyl)-3-thienyl]-1,3-dimethyl-1H-pyrazole-4-carboxamide of the formula

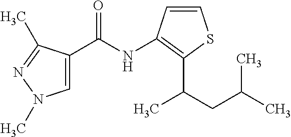 [SMILES: Cc1nn(C)cc1C(=O)Nc1ccsc1C(C)CC(C)C]

(known from EP-A 0 737 682)
(I-22) 3-(difluoromethyl)-N-[2-(1,3-dimethylbutyl)-3-thienyl]-1-methyl-1H-pyrazole-4-carboxamide of the formula

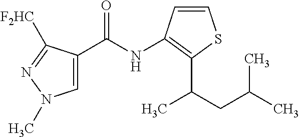 [SMILES: CC(C)CC(C)c1sccc1NC(=O)c1cn(C)nc1C(F)F]

(known from EP-A 0 737 682)
(I-23) 3-(trifluoromethyl)-N-[2-(1,3-dimethylbutyl)-3-thienyl]-1-methyl-1H-pyrazole-4-carboxamide of the formula

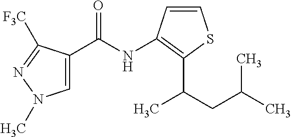 [SMILES: CC(C)CC(C)c1sccc1NC(=O)c1cn(C)nc1C(F)(F)F]

(known from EP-A 0 737 682)
(I-24) 3-(trifluoromethyl)-1-methyl-N-[2-(1,3,3-trimethylbutyl)-3-thienyl]-1H-pyrazole-4-carboxamide of the formula

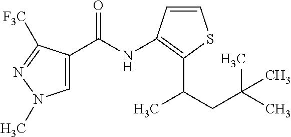 [SMILES: CC(CC(C)(C)C)c1sccc1NC(=O)c1cn(C)nc1C(F)(F)F]

(known from EP-A 0 737 682)
(I-25) N-[2-(1,3-dimethylbutyl)-3-thienyl]-2-chlorobenzamide of the formula

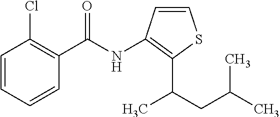 [SMILES: CC(C)CC(C)c1sccc1NC(=O)c1ccccc1Cl]

(known from EP-A 0 737 682)
(I-26) 2-chloro-N-[2-(1,3,3-trimethylbutyl)-3-thienyl]benzamide of the formula

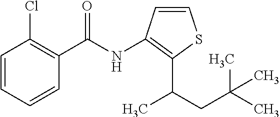 [SMILES: CC(CC(C)(C)C)c1sccc1NC(=O)c1ccccc1Cl]

(known from EP-A 0 737 682)
(I-27) 3-(difluoromethyl)-N-[2-(1,3-dimethylbutyl)-3-thienyl]-1-methyl-1H-pyrrole-4-carboxamide of the formula

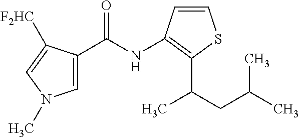 [SMILES: CC(C)CC(C)c1sccc1NC(=O)c1cn(C)cc1C(F)F]

(known from WO 02/38542)
(I-28) 3-(trifluoromethyl)-N-[2-(1,3,3-trimethylbutyl)-3-thienyl]-1-methyl-1H-pyrrole-4-carboxamide of the formula

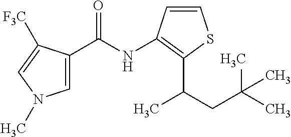 [SMILES: CC(CC(C)(C)C)c1sccc1NC(=O)c1cn(C)cc1C(F)(F)F]

(I-29) 3-(difluoromethyl)-N-[2-(1,3,3-dimethylbutyl)-3-thienyl]-1-methyl-1H-pyrrole-4-carboxamide of the formula

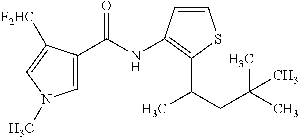 [SMILES: CC(CC(C)(C)C)c1sccc1NC(=O)c1cn(C)cc1C(F)F]

(I-30) 3-(trifluoromethyl)-N-[2-(1,3-dimethylbutyl)-3-thienyl]-1-methyl-1H-pyrrole-4-carboxamide of the formula

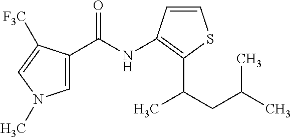 [SMILES: CC(C)CC(C)c1sccc1NC(=O)c1cn(C)cc1C(F)(F)F]

(known from WO 02/38542)

The active compounds according to the invention have very good fungicidal properties and, in seed treatment, are particularly suitable for controlling phytopathogenic fungi, such as Ascomycetes and Basidiomycetes. In seed treatment, the active compounds according to the invention are particularly suitable for controlling pyrenophora, rhizoctonia, tilletia and ustilago species.

Some pathogens causing fungal diseases which come under the generic names listed above may be mentioned by way of example, but not by way of limitation:

Pyrenophora species, such as, for example, P. allosuri, P. alternarina, P. avenae, P. bartramiae, P. bondarzewii, P. bromi, P. bryophila, P. buddleiae, P. bupleuri, P. calvertii, P. calvescens var. moravica, P. carthami, P. centranthi, P. cerastii, P. chengii, P. chrysanthemi, P. convolvuli, P. coppeyana, P. cytisi, P. dactylidis, P. dictyoides, P. echinopis, P. ephemera, P. eryngicola, P. erythrospila, P. euphorbiae, P. freticola, P. graminea, P. heraclei, P. hordei, P. horrida, P. hyperici, P. japonica, P. kugitangi, P. lithophila, P. lolii, P. macrospora, P. metasequoiae, P. minuartiae-hirsutae, P. moravica, P. moroczkovskii, P. muscorum, P. osmanthi, P. phlei, P. pimpinellae, P. pittospori, P. polytricha, P. polytricha, P. pontresinerisis, P. pulsatillae, P. raetica, P. rayssiae, P. rugosa, P. saviczii, P. schroeteri, P. scirpi, P. scirpicola, P. secalis, P. semeniperda, P. semiusta, P. seseli f. poterii, P. seseli, P. sobolevskii, P. subalpina, P. subantarctica, P. sudetica, P. syntrichiae, P. szaferiana, P. teres f maculata, P. teres subsp. graminea, P. teres, P. tetrarrhenae, P. tranzschelii, P. trzfolii, P. tritici-repentis, P. typhicola, P. ushuwaiensis, P. villosa, Rhizoctonia species, such as, for example, Rh. aerea, Rh. alba, Rh. alpina, Rh. anaticula, Rh. anomala, Rh. apocynacearum, Rh. arachnion, Rh. asclerotica, Rh. bataticola, Rh. borealis, Rh. callae, Rh. candida, Rh. carotae, Rh. cerealis, Rh. choussii, Rh. coniothecioides, Rh. dichotoma, Rh. dimorpha, Rh. endophytica var. filicata, Rh. endophytica, Rh. ferruginea, Rh. floccosa, Rh. fragariae, Rh. fraxini, Rh. fuliginea, Rh. fumigata, Rh. globularis, Rh. goodyerae-repentis, Rh. gossypii var. anatolica, Rh. gossypii, Rh. gracilis, Rh. grisea, Rh. hiemalis, Rh. juniperi, Rh. lamellifera, Rh. leguminicola, Rh. lilacina, Rh. lupini, Rh. macrosclerotia, Rh. melongenae, Rh. microsclerotia, Rh. monilioides, Rh. monteithiana, Rh. muneratii, Rh. nandorii, Rh. oryzae, Rh. otyzae-sativae, Rh. pallida, Rh. pini-insignis, Rh. praticola, Rh. quercus, Rh. ramicola, Rh. robusta, Rh. rubi, Rh. rubiginosa, Rh. sclerotica, Rh. solani, Rh. solani f. paroketea, Rh. solani forma specialis, Rh. solani var. cedri-deodarae, Rh. solani var. fuchsiae, Rh. solani var. hortensis, Rh. stahlii, Rh. subtilis var. nigra, Rh. subtilis, Rh. tomato, Rh. tuliparum, Rh. versicolor, Rh. zeae, Tilletia species, such as, for example, T. aegilopis, T. aegopogonis, T. ahmadiana, T. airina, T. ajrekari, T. alopecuri, T. anthoxanthi, T. apludae, T. arundinellae, T. asperifolia, T. asperitolioides, T. atacamensis, T. baldrati, T. bambusae, T. banarasae, T. bangalorensis, T. barclayana, T. biharica, T. bolayi, T. boliviensis, T. boutelouae, T. brachypodii, T. brachypodii-ramosi, T. brevifaciens, T. bromi., T. bromina, T. bromi-tectorum, T. brunkii, T. buchloeana, T. caries, T. cathcartae, T. cerebrina, T. chloridicola, T. controversa, T. controversa var. elymi, T. controversa var. prostrata, T. corona, T. cynosuri, T. dacamarae, T. deyeuxiae, T. digitariicola, T. durangensis, T. earlei, T. echinochloae f. foliicola, T. echinochloae, T. echinosperma, T. ehrhartae, T. eleusines, T. elymandrae, T. elymi, T. elymicola, T. elytrophori, T. eragrostidis, T. euphorbiae, T. fahrendorfii, T. festuca-octoflorana, T. foetida, T. fusca, T. fusca var. bromi-tectorum, T. fusca var. guyotiana, T. fusca var. patagonica, T. georfischeri, T. gigaspora, T. goloskokovii, T. haynaldiae, T. heterospora, T. holci, T. hordei var. spontanei, T. horrida, T. hyalospora var. cuzcoensis, T. hyparrheniae, T. indica, T. intermedia, T. iowensis, T. ixophori, T. koeleriae, T. kuznetzoviana, T. laevis, T. laguri, T. leptochloae, T. lepturi, T. lycuroides, T. maclaganii, T. macrotuberculata, T. madeirensis, T. makutensis, T. milli-vernalis, T. milli, T. montana, T. montemartinii, T. nanifica, T. narasimhanii, T. narayanaraoana, T. narduri, T. nigrifaciens, T. obscura-reticulata, T. oklahomae, T. okudairae, T. oplismeni-cristati, T. pachyderma, T. pallida, T. panici, T. panici-humilis, T. paradoxa, T. paspali, T. pennisetina, T. perotidis, T. phalaridis, T. poae, T. polypogonis, T. poonensis, T. prostrata, T. pulcherrima var. brachiariae, T. redfieldiae, T. rhei, T. rugispora, T. sabaudiae, T. salzmannii, T. savilei, T. scrobiculata, T. setariae, T. setariae-palmiflorae, T. setariicola, T. sphaerococca, T. sphenopi, T. sphenopodis, T. sterilis, T. taiana, T. texana, T. themedae-anatherae, T. themedicola, T. togwateei, T. trachypogonis, T. transiliensis, T. transvaalensis, T. tritici [var.] nanifica, T. tritici f. monococci, T. tritici var. controversa, T. tritici var. laevis, T. tritici-repentis, T. triticoides, T. tuberculata, T. vetiveriae, T. viennotii, T. vittata var. burmannii, T. vittata, T. walkeri, T. youngii, T. zundelii. Ustilago species, such as, for example, U. abstrusa, U. aegilopsidis, U. aeluropodis, U. affinis var. hilariae, U. agrestis, U. agropyrina, U. agrostis-palustris, U. ahmadiana, U. airae-caespitosae, U. alismatis, U. alopecurivora, U. alsineae, U. altilis, U. amadelpha var. glabriuscula, U. amphilophidis, U. amplexa, U. andropogonis-tectorum, U. aneilemae, U. anhweiana, U. anomala [var.] microspora, U. anomala var. avicularis, U. anomala var. carnea, U. anomala var. cordai, U. anomala var. muricata, U. anomala var. tovarae, U. anthoxanthi, U. apscheronica, U. arabidia-alpinae, U. arctagrostis, U. arctica, U. arenariae-bryophyllae, U. argentina, U. aristidarius, U. arundinellae-hirtae, U. asparagi-pygmaei, U. asprellae, U. avenae f.sp. perennans, U. avenae subsp. alba, U. avicularis, U. bahuichivoensis, U. barberi, U. beckeropsis, U. belgiana, U. bethelii, U. bicolor, U. bistortarum var. marginalis, U. bistortarum var. pustulata, U. bistortarum var. ustilaginea, U. borealis, U. bothriochloae, U. bothriochloae-intermediae, U. bouriqueti, U. braziliensis, U. brizae, U. bromi-arvensis, U. bromi-erecti, U. bromi-mollis, U. bromina, U. bromivora f. brachypodii, U. bromivora var. macrospora, U. bullata f. brachypodii-distachyi, U. bullata var. bonariensis, U. bullata var. macrospora, U. bungeana, U. calamagrostidis var. scrobiculata, U. calamagrostidis var. typica, U. calamagrostidis, U. cardamines, U. cariciphila, U. caricis-wallichianae, U. carnea, U. catherinae, U. caulicola, U. centrodomis, U. ceparum, U. cephalariae, U. chacoensis, U. chloridii, U. chloridionis, U. chrysopogonis, U. chubutensis, U. cichorii, U. cilinodis, U. clelandii, U. clintoniana, U. coloradensis, U. commelinae, U. compacta, U. concelata, U. condigna, U. consimilis, U. constantineanui, U. controversa, U. convertere-sexualis, U. cordal, U. coronariae, U. coronata, U. cortaderiae var. araucana, U. courtoisii, U. crus-galli var. minor, U. cryptica, U. curia, U. custanaica, U. cynodontis f. ovariicola, U. cynodontis, U. cyperi-lucidi, U. davisii, U. deccanii, U. decipiens, U. deformis, U. dehiscens, U. delicata, U. deyeuxiae, U. dianthorum, U. distichlidis, U. dubiosa, U. dumosa, U. earlei, U. echinochloae, U. ehrhartana, U. eleocharidis, U. eleusines, U. elymicola, U. elytrigiae, U. enneapogonis, U. epicampida, U. eragrostidis-japonicana, U. eriocauli, U. eriochloae, U. euphorbiae, U. fagopyri, U. festucae, U. festucarum, U. filamenticola, U. fingerhuthiae, U. flectens, U. flowersii, U. foliorum, U. formosana, U. fueguina, U. gageae, U. garcesi, U. gardneri, U. gaussenii, U. gigantispora, U. gigantospora, U. glyceriae, U. goyazana, U. gregaria, U. grossheimii, U. gunnerae, U. haesendocki var. chloraphorae, U. halophiloides var. vargasii, U. halophiloides, U. haynaldiae, U. heleochloae, U. helictotrichi, U. herteri var. bicolor, U. hierochloae-odoratae, U. hieronymi var. insularis, U. hieronymi var. minor, U. hilariicola, U. himalensis, U. hitchcockiana, U. holci-avenacei, U. holubii, U. hordei, U. hordei f.sp. avenae, U. hsuii, U. hyalino-bipolaris, U. hydropiperis, U. hyparrheniae, U. hypodytes f. congoensis, U. hypodytes f. sporoboli, U. hypodytes var. agrestis, U. idonea, U. imperatae, U. induta, U. inouyei, U. intercedens, U. iranica, U. isachnes, U. ischaemi-akoensis, U. ischaemi-anthephoroidis, U. ixiolirii, U. ixophori, U. jacksonii var. ventanensis, U. jacksonii, U. jaczevskyana var. sibirica, U. jaczevskyana var. typica, U. jaczevskyana, U. jagdishwari, U. jamalainenii, U. jehudana, U. johnstonii, U. kairamoi, U. kazachstanica, U. kenjiana, U. kweichowensis, U. kyllingae, U. lachrymae-jobi, U. lepyrodiclidis, U. lidii, U. liebenbergii, U. linderi, U. linearis, U. liroae, U. loliicola, U. longiflora, U. longiseti, U. longissima var. dubiosa, U. longissima var. paludificans, U. longissima var. typica, U. lupini, U. lychnidis-dioicae, U. lycoperdiformis, U. lyginiae, U. machili, U. magellanica, U. mariscana, U. maydis, U. megalospora, U. melicae, U. merxmuellerana, U. mesatlantica, U. michnoana, U. microspora var. paspalicola, U. microspora, U. microstegii, U. microthelis, U. milli, U. modesta, U. moehringiae, U. moenchiae-manticae, U. monermae, U. montagnei var. minor, U. morinae, U. morobiana, U. muehlenbergiae var. tucumanensis, U. muricata, U. muscari-botryoidis, U. nagornyi, U. nannfeldtii, U. nelsoniana, U. nepalensis, U. neyraudiae, U. nigra, U. nivalis, U. nuda, U. nuda var. hordei, U. nuda var. tritici, U. nyassae, U. okudairae, U. olida, U. olivacea var. macrospora, U. onopordi, U. onumae, U. opiziicola, U. oplismeni, U. orientalis, U. otophora, U. overeemii, U. pamirica, U. panici-geminati, U. panjabensis, U. pappophori var. magdalensis, U. pappophori, U. parasnathii, U. parodii, U. parvula, U. paspalidiicola, U. patagonica, U. penniseti var. verruculosa, U. perrara, U. persicariae, U. petrakii, U. phalaridis, U. phlei, U. phlei-pratensis, U. phragmites, U. picacea, U. pimprina, U. piperi [var.] rosulata, U. poae, U. poae-bulbosae, U. poae-nemoralis, U. polygoni-alati, U. polygoni-alpini, U. polygoni-punctati, U. polygoni-serrulati, U. polytocae, U. polytocae-barbatae, U. pospelovii, U. prostrata, U. pseudohieronymi, U. pueblaensis, U. puellaris, U. pulverulenta, U. raciborskiana, U. radians, U. ravida, U. rechingeri, U. reticulata, U. reticulispora, U. rhei, U. rhynchelytri, U. rwandensis, U. sabouriana, U. salviae, U. sanctae-catharinae, U. scaura, U. scillae, U. scitaminea var. sacchari-barberi, U. scitaminea var. sacchari-officinarum, U. scitaminea, U. scleranthi, U. scrobiculata, U. scutulata, U. secalis var. elymi, U. semenoviana, U. serena, U. serpens, U. sesleriae, U. setariae-mombassanae, U. shastensis, U. shimadae, U. silenes-inflatae, U. silenes-nutantis, U. sinkiangensis, U. sitanii, U. sleumeri, U. sonoriana, U. sorghi-stipoidei, U. spadicea, U. sparti, U. speculariae, U. spegazzinii var. agrestis, U. spegazzinii, U. spermophora var. orientalis, U. spermophoroides, U. spinulosa, U. sporoboli-indici, U. sporoboli-tremuli, U. stellariae, U. sterilis, U. stewartli, U. stipae, U. striaeformis f. agrostidis, U. striaeformis f. phlei, U. striaeformis f. poaeannuae, U. striaeformis f. poae-pratensis, U. striiformis f. hierochloes-odoratae, U. striiformis var. agrostidis, U. striiformis var. dactylidis, U. striiformis var. holci, U. striiformis var. phlei, U. striiformis var. poae, U. stygia, U. sumnevicziana, U. superba, U. sydowiana, U. symbiotica, U. taenia, U. taiana, U. tanakae, U. tenuispora, U. thaxteri, U. tinantiae, U. togata, U. tourneuxii, U. tovarae, U. trachyniae, U. trachypogonis, U. tragana, U. tragi, U. tragica, U. tragi-racemosi, U. trichoneurana, U. trichophora var. crus-galli, U. trichophora var. panici-frumentacei, U. triseti, U. tritici forma specialis, U. trochophora var. pacifica, U. tuberculata, U. tucumanensis, U. tumeformis, U. turcomanica var. prostrata, U. turcomanica var. typica, U. turcomanica, U. ugamica, U. ugandensis var. macrospora, U. underwoodii, U. urginede, U. urochloana, U. ustilaginea, U. utriculosa var. cordai, U. utriculosa var. reticulata, U. valentula, U. vavilovi, U. verecunda, U. verruculosa, U. versatilis, U. vetiveriae, U. violacea var. stellariae, U. violaceo-irregularis, U. violaceoverrucosa, U. williamsii, U. wynaadensis, U. zambettakisii, U. zernae.

The fact that the active compounds which can be used are well tolerated by plants at the concentrations required for controlling plant diseases permits a treatment of the seed. Accordingly, the active compounds according to the invention can be used as seed dressings.

A large part of the damage to crop plants which is caused by phytopathogenic fungi occurs as early as when the seed is attacked during storage and after the seed is introduced into the soil, during and immediately after germination of the plants. This phase is particularly critical since the roots and shoots of the growing plant are particularly sensitive and even minor damage can lead to the death of the whole plant. Protecting the seed and the germinating plant by the use of suitable compositions is therefore of particularly great interest.

The control of phytopathogenic fungi which damage plants post-emergence is carried out primarily by treating the soil and the above-ground parts of plants with crop protection agents. Owing to the concerns regarding a possible impact of crop protection agents on the environment and the health of man and animals, there are efforts to reduce the amount of active compounds applied.

The control of phytopathogenic fungi by treating the seeds of plants has been known for a long time and is subject-matter of continuous improvements. However, the treatment of seed frequently entails a series of problems which cannot always be solved in a satisfactory manner. Thus, it is desirable to develop methods for protecting the seed and the germinating plant which dispense with the additional application of crop protection agents after sowing or after the emergence of the plants or where additional applications are at least reduced. It is furthermore desirable to optimize the amount of active compound employed in such a way as to provide maximum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

The present invention therefore in particular also relates to a method for the protection of seed and germinating plants from attack by phytopathogenic fungi, by treating the seed with a composition according to the invention.

The invention likewise relates to the use of the compositions according to the invention for the treatment of seed for protecting the seed and the germinating plant from phytopathogenic fungi.

Furthermore, the invention relates to seed which has been treated with a composition according to the invention so as to afford protection from phytopathogenic fungi.

One of the advantages of the present invention is that the particular systemic properties of the compositions according to the invention mean that treatment of the seed with these compositions not only protects the seed itself, but also the resulting plants after emergence, from phytopathogenic fungi. In this manner, the immediate treatment of the crop at the time of sowing or shortly thereafter can be dispensed with.

Furthermore, it must be considered as advantageous that the mixtures according to the invention can also be employed in particular in transgenic seed.

The compositions according to the invention are suitable for protecting seed of any plant variety which is employed in agriculture, in the greenhouse, in forests or in horticulture. In particular, this takes the form of seed of cereals (such as wheat, barley, rye, millet and oats), maize, cotton, soya beans, rice, potatoes, sunflowers, beans, coffee, beet (for example sugar beet and fodder beet), peanuts, vegetables (such as tomatoes, cucumbers, onions and lettuce), lawns and ornamental plants. The treatment of seed of cereals (such as wheat, barley, rye and oats), maize and rice is of particular importance.

In the context of the present invention, the composition according to the invention is applied to the seed either alone or in a suitable formulation. Preferably, the seed is treated in a state which is stable enough to avoid damage during treatment. In general, the seed may be treated at any point in time between harvest and sowing. The seed usually used has been separated from the plant and freed from cobs, shells, stalks, coats, hairs or the flesh of the fruits. Thus, for example, it is possible to use seed which has been harvested, cleaned and dried to a moisture content of below 15% by weight. Alternatively, it is also possible to use seed which, after drying, has, for example, been treated with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which may have phytotoxic effects at certain application rates.

The compositions according to the invention can be applied directly, that is to say without comprising further components and without having been diluted. In general, it is preferable to apply the composition to the seed in the form of a suitable formulation. Suitable formulations and methods for the treatment of seed are known to the skilled worker and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compound combinations which can be used according to the invention can be converted into customary seed dressing formulations, such as solutions, emulsions, suspensions, powders, foams, slurries or other coating materials for seed, and also ULV formulations.

These formulations are prepared in a known manner by mixing the active compounds or active compound combinations with customary additives, such as, for example, customary extenders and also solvents or diluents, colorants, wetting agents, dispersants, emulsifiers, defoamers, preservatives, secondary thickeners, adhesives, gibberellins and optionally water as well.

Suitable colorants that may be present in the seed dressing formulations of the invention include all colorants customary for such purposes. Use may be made both of pigments, of sparing solubility in water, and of dyes, which are soluble in water. Examples that may be mentioned include the colorants known under the designations rhodamine B, C.I. Pigment Red 112, and C.I. Solvent Red 1.

Suitable wetting agents that may be present in the seed dressing formulations of the invention include all substances which promote wetting and are customary in the formulation of active agrochemical substances. With preference it is possible to use alkylnaphthalene-sulphonates, such as diisopropyl- or diisobutylnaphthalene-sulphonates.

Suitable dispersants and/or emulsifiers that may be present in the seed dressing formulations of the invention include all nonionic, anionic, and cationic dispersants which are customary in the formulation of active agrochemical substances. With preference, it is possible to use nonionic or anionic dispersants or mixtures of nonionic or anionic dispersants. Particularly suitable nonionic dispersants are ethylene oxide-propylene oxide block polymers, alkylphenol polyglycol ethers, and tristyrylphenol polyglycol ethers, and their phosphated or sulphated derivatives. Particularly suitable anionic dispersants are lignosulphonates, polyacrylic salts, and arylsulphonate-formaldehyde condensates.

Suitable defoamers that may be present in the seed dressing formulations of the invention include all foam-inhibiting substances which are customary in the formulation of active agrochemical substances. With preference it is possible to use silicone defoamers and magnesium stearate.

Suitable preservatives that may be present in the seed dressing formulations of the invention include all substances which can be used for such purposes in agrochemical compositions. By way of example, mention may be made of dichlorophen and benzyl alcohol hemiformal.

Suitable secondary thickeners that may be present in the seed dressing formulations of the invention include all substances which can be used for such purposes in agrochemical compositions. Preferred suitability is possessed by cellulose derivatives, acrylic acid derivatives, xanthan, modified clays, and highly disperse silica.

Suitable adhesives that may be present in the seed dressing formulations of the invention include all customary binders which can be used in seed dressing. With preference, mention may be made of polyvinylpyrrolidone, polyvinyl acetate, polyvinyl alcohol and tylose.

Suitable gibberellins that may be present in the seed dressing formulations of the invention include preferably substances of the formula

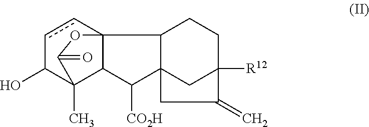

in which
R¹² represents hydrogen or hydroxyl and
the dashed line indicates that in the position of the ring either a C—C single bond or a C═C double bond is present.

Examples that may be mentioned of gibberellins of the formula (II) include the following:

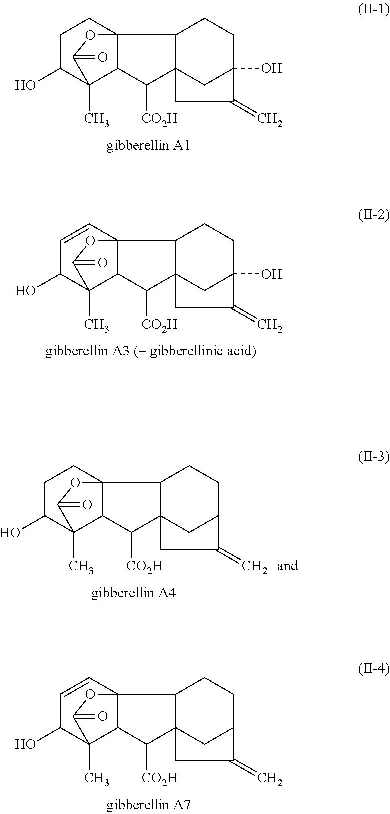 [SMILES: C=C1CC23C[C@@]1(O)CCC2C12CCC(O)C(C)(C(=O)O1)C2C3C(=O)O ; C=C1CC23C[C@@]1(O)CCC2C12C=CC(O)C(C)(C(=O)O1)C2C3C(=O)O ; C=C1CC23CC1CCC2C12CCC(O)C(C)(C(=O)O1)C2C3C(=O)O ; C=C1CC23CC1CCC2C12C=CC(O)C(C)(C(=O)O1)C2C3C(=O)O]

Particular preference is given to gibberellic acid of the formula (II-2).

The gibberellins of the formula (II) are known (cf. R. Wegler “Chemie der Pflanzen-schutz- and Schädlingsbekämpfungsmittel”, Volume 2, Springer Verlag, Berlin-Heidelberg-New York, 1970, pages 401-412).

The seed dressing formulations to be used according to the invention may be used directly or after dilution with water beforehand to treat seed of any of a very wide variety of types. For instance, the concentrates or the preparations obtainable therefrom by dilution with water may be used to dress the seed of cereals, such as wheat, barley, rye, oats, and triticale, and also the seed of maize, rice, oilseed rape, peas, field beans, cotton, sunflowers, and beets, or else vegetable seed of any of a very wide variety of kinds. The suitable seed dressing formulations of the invention or their dilute preparations may also be used to dress seed of transgenic plants. In this context, synergistic effects may also arise in interaction with the substances formed by expression.

Suitable mixing equipment for treating seed with the seed dressing formulations to be used according to the invention or the preparations prepared from them by adding water includes all mixing equipment which can commonly be used for dressing. The specific procedure adopted when dressing comprises introducing the seed into a mixer, adding the particular desired amount of seed dressing formulation, either as it is or following dilution with water beforehand, and carrying out mixing until the formulation is uniformly distributed on the seed. Optionally, a drying operation follows.

The application rate of the seed dressing formulations to be used according to the invention may be varied within a relatively wide range. It depends on the respective content of the active compounds in the formulations and on the seed. In general, the application rates of active compound combination are between 0.001 and 50 g per kilogram of seed, preferably between 0.01 and 15 g per kilogram of seed.

The good fungicidal action of the active compounds to be used according to the invention in the treatment of seed is demonstrated by the examples below.

### USE EXAMPLES

### Example A

### Rhizoctonia solani Test (Cotton)/Seed Treatment

The active compounds are applied as dry seed dressing. This is prepared by extending the active compounds in question with ground minerals to give a finely pulverulent mixture which ensures uniform distribution on the seed surface.

To dress the seed, the seed together with the seed dressing is shaken for 3 minutes in a closed glass bottle.

2×50 seed corns are sown in standardized soil in seed trays. Perlite infected with Rhizoctonia solani (5 ml/seed tray) is scattered between the seed and covered with Lecaton, and the seeds are then cultivated in a greenhouse at a temperature of about 22° C. and 15 hours of light per day.

After 7-8 days, all the emerged and diseased plants are evaluated. The effect is calculated by the method of Abbot. 0% means an efficacy which corresponds to that of the control, whereas an efficacy of 100% means that no infection is observed.

TABLE A
Rhizoctonia solani test (cotton)/seed treatment

| | Application | |
|---|---|---|
| | rate of | |
| | active | |
| | compound | |
| | in g/100 | Effi- |
| | kg of | cacy |
| Active compound | seed | in % |
| 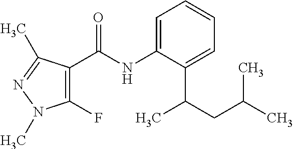 [SMILES: Cc1nn(C)c(F)c1C(=O)Nc1ccccc1C(C)CC(C)C] | 50 | 100 |
| 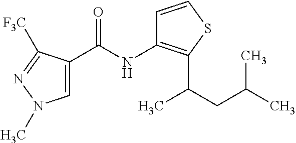 [SMILES: CC(C)CC(C)c1sccc1NC(=O)c1cn(C)nc1C(F)(F)F] | 50 | 100 |

### Example B

### Pyrenophora graminea Test (Barley)/Seed Treatment/Outdoor Trial

Cereal species: Summer barley
Plot size: 2 m²
Amount of seed per plot: 40 g
Number of repetitions: 3
Development stage at the time of evaluation: Begin of heading

The active compounds are applied as a commercial formulation.

For dressing, the preparation is initially charged in a dressing beaker and placed on a dressing shaker. The infected seed is added and shaken for about 2-3 min, until homogenization is achieved.

After preparing the soil in a customary manner, outdoor sowing is carried out at a time which favours infection by the disease.

Evaluation is carried out at a time when all the disease symptoms are clearly present. 0% means an efficacy which corresponds to that of the control, whereas an efficacy of 100% means that no infection is observed.

TABLE B
Pyrenophora graminea test (barley)/seed treatment/outdoor trial

| | Application | |
|---|---|---|
| | rate of | |
| | active | |
| | compound | |
| | in g/100 | Effi- |
| | kg of | cacy |
| Active compound | seed | in % |
| 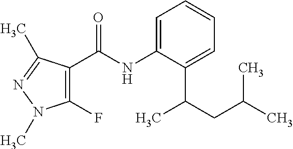 [SMILES: Cc1nn(C)c(F)c1C(=O)Nc1ccccc1C(C)CC(C)C] | 10 | 97 |
| 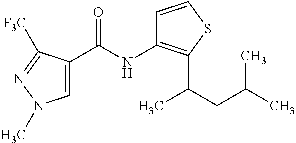 [SMILES: CC(C)CC(C)c1sccc1NC(=O)c1cn(C)nc1C(F)(F)F] | 10 | 97 |

The infection in the summer barley cultivar “Frisia” was 10.1%.

### Example C

### Rhizoctonia solani Test (Rice)/Seed Treatment

Rice seed (cultivar Koshihikari) is soaked at 15° C. in distilled water for 10 days, transferred into fresh water and, whilst being ventilated, soaked at 32° C. for 1 day. The seed is then dried at room temperature for a number of hours. 7.6 mg of the test substance are dissolved in 200 μl of acetone in a test vessel, an aliquot of seed (corresponding to 3.8 g of dry seed) is added, the samples mixed and the acetone is aspirated. The seed is sown on soil in a plastic vessel having a diameter of 7.5 cm and cultivated in an incubator at 32° C. with high atmospheric humidity in the dark for 3 days. The seedlings are then cultivated in a greenhouse at on average 21° C. for 2 weeks. In each case 5 seedlings are transferred into 5 plastic vessels having a diameter of 12 cm and cultivated in a greenhouse at on average 25° C. for 5-6 weeks. Mycelia of Rhizoctonia solani which had been grown on sterile barley corns are placed at the foot of the rice plant (5 cm from the soil surface) and incubated in a glass chamber with high atmospheric humidity at on average 27° C.

Seven days after the inoculation, the degree of infection is evaluated and the efficacy is calculated.

Criteria for assessing the infection with the disease (disease rate)

| Disease rate | Vertical extension of the plant disease (cm) |
|---|---|
| 0 | 0 |
| 0.5 | <1.5 |
| 1 | 3-5 |
| 2 | 6-11 |
| 3 | 12 |
| 4 | 13-23 |
| 5 | >24 |

$Efficacy ( \% ) = 1 \frac{Disease rate of the infected plant}{Disease rate of the untreated control} 100$

TABLE C
Rhizoctonia solani test (rice)/seed dressing

| | Application | |
|---|---|---|
| | rate of | |
| | active | |
| | compound | |
| | in g/100 | Effi- |
| | kg of | cacy |
| Active compound | seed | in % |
| 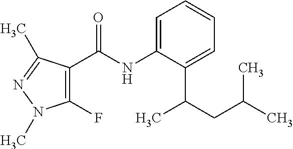 [SMILES: Cc1nn(C)c(F)c1C(=O)Nc1ccccc1C(C)CC(C)C] | 200 | 91 |

### Example D

### Ustilago nuda Test (Barley)/Seed Treatment/Outdoor Trial

Cereal species: Summer barley
Amount of seed per plot: 40 g
Plot size: 2 m²
Number of repetitions: 3
Development stage: Begin to middle of blossom

The active compounds are applied as a commercial formulation.

For dressing, the preparation is initially charged in a dressing beaker and placed on a dressing shaker. The infected seed is added and shaken for about 2-3 min, until homogenization is achieved.

After preparing the soil in a customary manner, outdoor sowing is carried out at a time which favours infection by the disease.

Evaluation is carried out at a time when all the disease symptoms are clearly present. 0% means an efficacy which corresponds to that of the control, whereas an efficacy of 100% means that no infection is observed.

TABLE D
Ustilago nuda test (barley)/seed treatment/outdoor trial

| | Application | |
|---|---|---|
| | rate of | |
| | active | |
| | compound | |
| | in g/100 | Effi- |
| | kg of | cacy |
| Active ingredient | seed | in % |
| 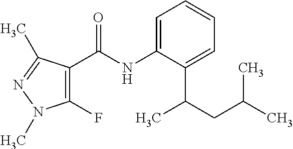 [SMILES: Cc1nn(C)c(F)c1C(=O)Nc1ccccc1C(C)CC(C)C] | 10 | 100 |
| 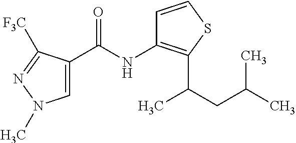 [SMILES: CC(C)CC(C)c1sccc1NC(=O)c1cn(C)nc1C(F)(F)F] | 10 | 99 |

The infection in the summer barley cultivar “Frisia” was 10.1%.

1.-6. (canceled)

7. A method of dressing seed for controlling Rhizoctonia solani comprising: treating said seed with N-[2-(1,3-dimethylbutyl)phenyl]-5-fluoro-1,3-dimethyl-1H-pyrazole-4-carboxamide (I-2) at a rate of from 1 g to 50 g per 100 kg of seed, wherein said seed is rice seed, and wherein Rhizoctonia solani is controlled.

8. A method of dressing seed for controlling Rhizoctonia solani comprising: treating said seed with N-[2-(1,3-dimethylbutyl)phenyl]-5-fluoro-1,3-dimethyl-1H-pyrazole-4-carboxamide (I-2) at a rate of from 1 g to 50 g per 100 kg of seed, wherein said seed is cotton seed, and wherein Rhizoctonia solani is controlled.

9.-14. (canceled)

15. A method for controlling Rhizoctonia solani on seed comprising: treating said seed with N-[2-(1,3-dimethylbutyl)phenyl]-5-fluoro-1,3-dimethyl-1H-pyrazole-4-carboxamide (I-2) at a rate of from 1 g to 50 g per 100 kg of seed wherein said seed is cotton seed or rice seed, and wherein Rhizoctonia solani is controlled.

16.-21. (canceled)

22. The method according to claim 15, wherein said seed is treated at a rate of 50 g per 100 kilograms of seed.

23. The method according to claim 7, wherein said seed is treated with N-[2-(1,3-dimethylbutyl)phenyl]-5-fluoro-1,3-dimethyl-1H-pyrazole-4-carboxamide (I-2) at a rate of from 10 g to 50 g per 100 kg of seed.

24. The method according to claim 7, wherein said seed is treated with N-[2-(1,3-dimethylbutyl)phenyl]-5-fluoro-1,3-dimethyl-1H-pyrazole-4-carboxamide (I-2) at a rate of from 10 g to 50 g per 100 kg of seed.
